(19) United States
(12) Patent Application Publication
(10) Pub. No.: US 2022/0401650 A1
(43) Pub. Date: Dec. 22, 2022

(54) DRUG DELIVERY DEVICE HAVING SHOCK ABSORBER

(71) Applicant: AMGEN INC., Thousand Oaks, CA (US)

(72) Inventors: Kasper Poder, Tikøb (DK); David Lavmand Muller, Thousand Oaks, CA (US); Peter Werner Hansen, Birkerod (DK); Lars Eilertsen, Søborg (DK)

(21) Appl. No.: 17/841,845

(22) Filed: Jun. 16, 2022

Related U.S. Application Data
(60) Provisional application No. 63/211,904, filed on Jun. 17, 2021.

(51) Int. Cl. A61M 5/20; A61M 5/24; A61M 5/315

(52) U.S. Cl. CPC A61M 5/2033; A61M 5/24; A61M 5/31505; A61M 2005/2418

(57) ABSTRACT

A drug delivery device is provided, including a housing, a drug storage container, a plunger, a plunger biasing member, a releaser, and a shock absorber. The housing defines a longitudinal axis and has an opening. The drug storage container includes a barrel, a stopper, and a delivery member, where the stopper is movably positioned within the barrel. The delivery member is positioned at a distal end of the barrel and has an insertion end configured to extend at least partially through the opening during a delivery state. The plunger is moveable toward the distal end of the drug storage container to engage the stopper and expel a drug from the drug storage container through the delivery member. The plunger biasing member is coupled with the plunger and configured to urge the plunger toward the distal end of the drug storage container. The releaser member has a first position wherein the releaser member prevents the plunger from moving into the delivery state and a second position wherein the releaser member does not prevent the plunger from moving into the delivery state. The shock absorber is configured to absorb an impact force and prevent unintended movement of the releaser member.

### FIELD OF DISCLOSURE

The present disclosure generally relates to drug delivery devices and, more particularly, devices for automatically injecting a drug into a patient.

### BACKGROUND

A general aversion to exposed needles, as well as health and safety issues, have led to the development of drug delivery devices which conceal a needle or other insertion member prior to use and which automate various aspects of an injection process. Such devices offer a variety of benefits as compared with traditional forms of drug delivery including, for example, delivery via a conventional syringe.

A drug delivery device may incorporate various mechanisms to implement various automated or semi-automated features. Such features may include automatically covering a needle in a pre-delivery and/or post-delivery state, automatically inserting a needle and/or a cannula into a user, automatically activating a drive mechanism, automatically indicating to the user that drug delivery is complete, locking a guard in a needle covering position after drug delivery is complete, among other features. Certain such features are activated by the application of an external force, for example, by a user. Such features may be prone to premature or inadvertent activation in cases where the drug delivery device subjected to a sudden unintended force or motion during manufacture, transport, storage, and/or other handling of the device.

For example, a drug delivery device may experience a substantial impulse force if it is dropped from a height and strikes a stationary surface such as the ground. The impulse force has the potential to prematurely activate the automated or semi-automated features and/or cause structural damage to the drug delivery device. The likelihood of such problems is increased if the drug delivery device has recently been removed from cold storage, which is required for drug delivery devices containing certain drugs. In a cold state, various components of the drug delivery device may be relatively brittle and thus vulnerable to fracture or damage as the result of a sudden impact.

The present disclosure sets forth drug delivery devices embodying advantageous alternatives to existing drug delivery devices, and device housing features, and that may address one or more of the challenges or needs mentioned herein.

### SUMMARY

One aspect of the present disclosure provides a drug delivery device including a housing, a drug storage container, a plunger, a plunger biasing member, a releaser, and a shock absorber. The housing may define a longitudinal axis and has an opening. The drug storage container may include a barrel, a stopper, and a delivery member, where the stopper is movably positioned within the barrel. The delivery member may be positioned at a distal end of the barrel and has an insertion end configured to extend at least partially through the opening during a delivery state. The plunger may be moveable toward the distal end of the drug storage container to engage the stopper and expel a drug from the drug storage container through the delivery member. The plunger biasing member may be coupled with the plunger and configured to urge the plunger toward the distal end of the drug storage container. The releaser member may have a first position wherein the releaser member prevents the plunger from moving into the delivery state and a second position wherein the releaser member does not prevent the plunger from moving into the delivery state. The shock absorber may be configured to absorb an impact force and prevent unintended movement of the releaser member.

The housing may include a tubular housing and a rear cap operably coupled with each other, and the shock absorber may include the rear cap. The rear cap may be movable with respect to the tubular housing.

The shock absorber may include a snap ring configured to permit relative movement between the rear cap and the tubular housing.

The shock absorber may include an annular ridge configured to be received by the snap ring. The snap ring may includes a ramped surface configured to permit the rear cap to move in a distal direction upon application of the impact force and to urge the rear cap to move in a proximal direction after dissipation of the impact force. The ramped surface may be defined by a plurality of longitudinal ribs.

The device may include a buffer gap between the rear cap and the tubular housing. The buffer gap may define a distance between the rear cap and the tubular housing.

The device may include a plunger guide configured to operatively couple the rear cap and the tubular housing. The plunger guide may define the annular ridge received by the snap ring of the rear cap and the plunger guide may further define a second annular ridge configured to be received by a second annular ring.

The plunger guide may be configured to operatively couple the tubular housing and the rear cap, and the plunger guide may define the annular ridge.

The drug delivery device may be an autoinjector but is not limited to being an autoinjector.

The tubular housing may define a generally cylindrical shape. The tubular housing may define a non-cylindrical shape, such as a generally oblong or oval shape.

The tubular housing and the rear cap may be defined by a single, monolithic structure and the shock absorber may include a flexible or compressible portion coupling the tubular housing and the rear cap.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a perspective view of an exemplary drug delivery device in accordance with various embodiments, with a shock absorber configured to absorb an impact force;

FIG. 2 is a cross-sectional view of the drug delivery device in FIG. 1;

FIG. 3A is a close-up cross-sectional view of portions of the drug delivery device in FIG. 1, where the shock absorber is in a first position where the device is not experiencing an impact event or the after-effects thereof;

FIG. 3B is a close-up cross-sectional view of the portions of the drug delivery device in FIG. 1, where the housing is in a second position during an impact event or the after-effects thereof;

FIG. 4A is an exploded assembly view of a portion, namely the drive mechanism, of the drug delivery device in FIG. 1;

FIG. 4B is an exploded assembly view of the drug delivery device in FIG. 1;

FIG. 5 is a cross-sectional view of a rear cap that may be utilized as part of a shock absorber in accordance with various aspects of a drug delivery device;

FIG. 6 is a cross-sectional view of another rear cap that may be utilized as part of a shock absorber in accordance with various aspects of a drug delivery device;

FIG. 7 is a cross-sectional view of yet another rear cap that may be utilized as part of a shock absorber in accordance with various aspects of a drug delivery device;

FIG. 8 is a cross-sectional view of another rear cap that may be utilized as part of a shock absorber in accordance with various aspects of a drug delivery device; and

FIG. 9A is a cross-sectional view of portions of a housing and plunger guide that may be utilized as part of a shock absorber in accordance with various aspects of a drug delivery device, where the housing is in a first position where the device is not experiencing an impact event or the after-effects thereof; and

FIG. 9B is a cross-sectional view of the portions of the housing and plunger guide shown in FIG. 9A, where the housing is in a second position during an impact event or the after-effects thereof.

### DETAILED DESCRIPTION

The present disclosure generally relates to drug delivery devices operable by a user for administering a drug, or in the case where a patient is the user, self-administering a drug. The drug delivery device may include a housing, a drug storage container, a plunger, a plunger biasing member, a releaser, and a shock absorber. The housing may define a longitudinal axis and has an opening. The drug storage container may include a barrel, a stopper, and a delivery member, where the stopper is movably positioned within the barrel. The delivery member may be positioned at a distal end of the barrel and has an insertion end configured to extend at least partially through the opening during a delivery state. The plunger may be moveable toward the distal end of the drug storage container to engage the stopper and expel a drug from the drug storage container through the delivery member. The plunger biasing member may be coupled with the plunger and configured to urge the plunger toward the distal end of the drug storage container. The releaser member may have a first position wherein the releaser member prevents the plunger from moving into the delivery state and a second position wherein the releaser member does not prevent the plunger from moving into the delivery state. The shock absorber may be configured to absorb an impact force and prevent unintended movement of the releaser member.

The presently disclosed shock absorber may allow the cap or certain portion(s) thereof to move with respect to other components of the device (e.g., the housing) to diminish or dampen at least of some of the mechanical effects of the externally applied force including, for example, reducing an acceleration and/or deceleration caused by the externally applied force. Accordingly, the shock absorber may prevent or inhibit the activation of one or more automated or semi-automated features included in the drug delivery device including, for example, a drive mechanism for expelling a drug, a releaser, among others. In addition, the presently disclosed shock absorbing features may prevent or inhibit damage to the drug delivery device, including the cap, that may otherwise result from the externally applied force. For example, the shock absorber may diminish the likelihood of fractures or cracks forming in the cap and/or other portions of the drug delivery device if a user accidentally drops the drug delivery device after removing it from cold storage. These and other advantages will be apparent to one of ordinary skill in the art reviewing the present disclosure.

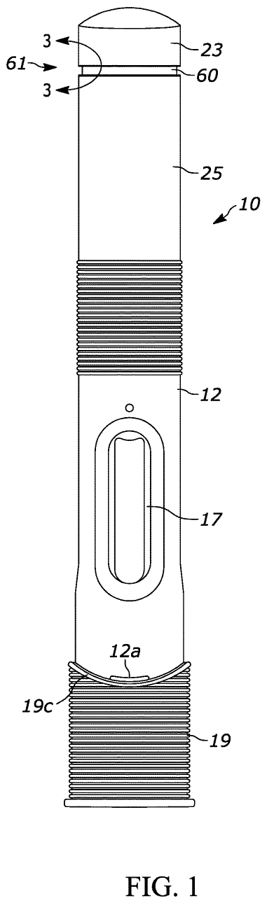

FIGS. 1-3 illustrate several views of an embodiment of a drug delivery device 10 for delivering a drug, which may also be referred to herein as a medicament or drug product. The drug may be, but is not limited to, various biologics such as peptides, peptibodies, or antibodies. The drug may be in a fluid or liquid form, although the present disclosure is not limited to a particular state.

Various implementations and configurations of the drug delivery device 10 are possible. The present embodiment of the drug delivery device 10 is configured as a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). The exemplary the drug delivery devices shown in the figures may take the form of an autoinjector or pen-type injector, and, as such, may be held in the hand of the user over the duration of drug delivery, but may also or alternatively be suitable for other drug delivery devices and/or configurations.

The configuration of various components included in the drug delivery device 10 may depend on the operational state of the drug delivery device 10. The drug delivery device 10 may have a storage state, a pre-delivery state, a delivery or dosing state, and a post-delivery state, although fewer or more states are also possible. For example, each state may have several sub-states or stages. The storage state may correspond to the configuration of the drug delivery device 10 in FIGS. 1-3, where the delivery device includes a removable cap in a storage position. In some embodiments, the storage state may exist in the time between when the drug delivery device 10 leaves a manufacturing facility and when a patient or other user removes the removable cap. The pre-delivery stage may correspond to the configuration of the drug delivery device 10 after the removable cap has been removed but prior to activation of a drive mechanism by the user. This may include the moments in time after the user has removed the removable cap, while the user is first positioning the drug delivery device 10 against the injection site, but before dosing has begun. The delivery state may correspond to the configuration of the drug delivery device 10 while drug delivery, also referred to herein as dosing, is in progress. The post-delivery state may correspond to the configuration of the drug delivery device 10 after drug delivery is complete and/or when a stopper is arranged in an end-of-dose position in a drug storage container.

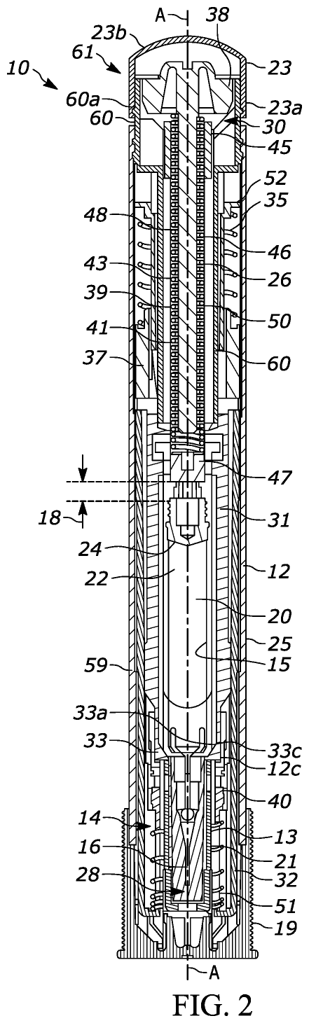

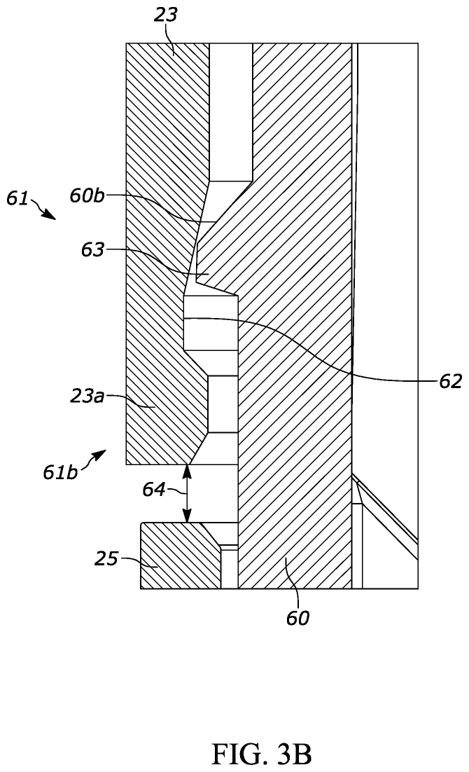

Referring to FIGS. 1-4B, the drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis A between a proximal end and a distal end. An opening 14 (FIG. 2) may be formed in the distal end (bottom end in FIGS. 1-3) to permit an insertion end 28 of a delivery member 16 to extend outside of the housing 12. A transparent or semi-transparent inspection window 17 may be positioned in a wall of the housing 12 to permit a user to view component(s) inside the drug delivery device 10, including a drug storage container 20. Viewing the drug storage container 20 through the window 17 may allow a user to confirm that drug delivery is in progress and/or complete. A removable cap 19 may cover the opening 14 at the distal end of the device prior to use of the drug delivery device 10, and, in some embodiments, may include a gripper 13 configured to assist with removing a removable sterile barrier 21 (e.g., a rigid needle shield (RNS), a non-rigid needle shield (nRNS), etc.) mounted on the insertion end 28 of the delivery member 16. The gripper 13 may include one or more inwardly protruding barbs or arms that frictionally or otherwise mechanically engage the removable sterile barrier 21 to pull the removable sterile barrier 21 with the removable cap 19 when the user separates the removable cap 19 from the housing 12. Thus, removing the removable cap 19 has the effect of removing the removable sterile barrier 21 from the delivery member 16.

In some embodiments, the housing 12 may include two separate and interconnected structures: a rear end cap 23 (e.g., a rear cover) at the proximal end of the drug delivery device 10; and a tubular housing 25 extending substantially completely along the length of the drug delivery device 10 and defining the opening 14. Additionally or alternatively, the housing 12 may include fewer or more components, such as a two-piece tubular housing having front and rear portions. The tubular housing 25 may have a hollow and generally cylindrical or tubular shape, and the rear end cap 23 may have a generally hemispherical shape or a hollow cylindrical shape with an open end and a closed off end. In some embodiments, the rear end cap 23 and the tubular housing 25, and any components to be positioned therein, may be assembled together to define different sub-assemblies. In alternative embodiments, the housing 12 may be constructed in one piece, such that the housing 12 is defined by a single, monolithic structure that integrates a rear cap and tubular housing in a single component. In such a one-piece housing embodiment, the housing may include a flexible or compressible portion that serves as the shock absorber.

The drug storage container 20 is disposed within an interior space of the housing 12 and is configured to contain a drug. The drug storage container 20 may be pre-filled and shipped, e.g., by a manufacturer, to a location where the drug storage container 20 is combined with a remainder of the drug delivery device 10. For example, the drug 22 may be distributed and/or provided to patients in more than one use case, such as a as a pre-filled syringe or as an autoinjector including a pre-filled syringe. By utilizing the same or similar syringe components in either case, at least some of above steps such as filling, labeling, packaging, shipping, and distribution may be streamlined or simplified for two different use cases. As another example, in the event that multiple use cases utilize some or all of the same syringe components, some regulatory pathways to marketing and/or distributing the drug may be streamlined and/or simplified for at least one of the multiple use cases.

The drug storage container 20 may include a rigid wall defining an internal bore or reservoir. The wall may be made of glass or plastic. A stopper 24 may be moveably disposed in the drug storage container 20 such that it can move in a distal direction along the longitudinal axis A between proximal end and a distal end of the drug storage container 20. The stopper 24 may be constructed of rubber or any other suitable material. The stopper 24 may slidably and sealingly contact an interior surface 15 of the wall of the drug storage container 20 such that the drug 22 is prevented or inhibited from leaking past the stopper 24 when the stopper 24 is in motion. Distal movement of the stopper 24 expels the drug 22 from the reservoir of the drug storage container 20 into the delivery member 16. The proximal end of the drug storage container 20 may be open to allow a plunger 26 to extend into the drug storage container 20 and push the stopper 24 in the distal direction. In the present embodiment, the plunger 26 and the stopper 24 are initially spaced from each other by a gap 18. Upon activation of a drive mechanism 30, the plunger 26 moves in the distal direction to close the gap 18 and comes into contact with the stopper 24. Subsequent distal movement of the plunger 26 drives the stopper 24 in the distal direction to expel the drug 22 from the drug storage container 20. In alternative embodiments, the stopper 24 and the plunger 26 may initially be in contact with one another or coupled to one another, e.g., via a threaded coupling, such that they move together jointly from the start of movement of the plunger 26. Once the stopper 24 is in motion, it may continue to move in the distal direction until it contacts a proximally-facing portion of the interior surface 15 of the wall of the drug storage container 20. This position of the stopper 24 may be referred to as the end-of-dose or end-of-delivery position, and may correspond to when delivery of the drug 22 to the patient is complete or substantially complete.

In some embodiments, a volume of the drug 22 included in the reservoir of the drug storage container 20 may be equal to 1 mL, or equal to approximately (e.g., ±10%) 1 mL, or equal to 2.5 mL, or equal to approximately (e.g., ±10%) 2.5 mL, or equal to 3 mL, or equal to approximately (e.g., ±10%) 3 mL, or less than or equal to approximately (e.g., ±10%) 1 mL, or less than or equal to approximately (e.g., ±10%) 2 mL, or less than or equal to approximately (e.g., ±10%) 3 mL, or less than or equal to approximately (e.g., ±10%) 4 mL, or less than approximately (e.g., ±10%) 5 mL, or less than or equal to approximately (e.g., ±10%) 10 mL, or within a range between approximately (e.g., ±10%) 1-10 mL, or within a range between approximately (e.g., ±10%) 1-5 mL, or within a range between approximately (e.g., ±10%) 1-4 mL, or within a range between approximately (e.g., ±10%) 1-3 mL, or within a range between approximately (e.g., ±10%) 1-2.5 mL.

The delivery member 16 is connected or operable to be connected in fluid communication with the reservoir of the drug storage container 20. A distal end of the delivery member 16 may define the insertion end 28 of the delivery member 16. The insertion end 28 may include a sharpened tip of other pointed geometry allowing the insertion end 28 to pierce the patient's skin and subcutaneous tissue during insertion of the delivery member 16. The delivery member 16 may be hollow and have an interior passageway. One or more openings may be formed in the insertion end 28 to allow drug to flow out of the delivery member 16 into the patient.

In one embodiment, the drug storage container 20 may be a pre-filled syringe and has a staked, hollow metal needle for the delivery member 16. Here, the needle is fixed relative to the wall of the drug storage container 20 and may be in permanent fluid communication with the reservoir of the drug storage container 20. In other embodiments, the needle may be coupled to the drug storage container 20 via a Luer Lock or other suitable connection. In yet other embodiments, the drug storage container 20 may be a needle-less cartridge, and, as such, initially may not be in fluid communication with the delivery member 16. In such embodiments, the drug storage container 20 may move toward a proximal end of the delivery member 16, or vice versa, during operation of the drug delivery device 10 such that the proximal end of the delivery member 16 penetrates through a septum covering an opening in the drug storage container 20 thereby establishing fluid communication between the reservoir of the drug storage container 20 and the delivery member 16.

The device may also include a container holder 33 configured to secure the drug storage container 20 with respect to the housing 12, such as by preventing distal movement of the drug storage container 20 during actuation of the plunger. The container holder 33 may include a plurality of flanges 33c that each include an arcuate, sloped surface 33a that substantially matches the arcuate shape of a shoulder portion of the drug storage container 20. As a more specific example, when the drug storage container 20 is inserted within the container holder 33, the flanges 33c cooperate to support the shoulder portion and limit the travel of the drug storage container 20 in the distal direction. The housing 12 may include a plurality of lock slots 12c that each receive respective flanges 33c of the container holder 33 to prevent and/or restrict relative movement between the respective components 12, 33. As a result, when fully assembled the storage container 20, the container holder 33, and the housing 12 are all substantially or completely fixed with respect to each other.

The drug delivery device 10 may further include a guard mechanism for preventing contact with the insertion end 28 of the delivery member 16 when the drug delivery device 10 is not being used to administer an injection. The guard mechanism may include a guard member 32 moveably disposed at the distal end of the housing 12 adjacent to the opening 14. The guard member 32 may have a hollow and generally cylindrical or tubular shape centered generally about the longitudinal axis A, and may have a proximal end received within the housing 12. The guard member 32 may be configured to move relative to the housing 12 between an extended position wherein a distal end of the guard member 32 extends through the opening 14 in the housing 12 and a retracted position wherein the distal end of the guard member 32 is retracted, fully or partially, into the opening 14 in the housing 12. Additionally or alternatively, the guard member 32 may be configured to move from the retracted position to the extended position. When moving from the extended position to the retracted position, the guard member 32 may translate linearly in the proximal direction; and when moving from the retracted position to the extended position, the guard member 32 may translate linearly in the distal direction. In at least the extended position, the guard member 32 may extend beyond and surround the insertion end 28 of the delivery member 16. In embodiments where the delivery member 16 protrudes from the opening 14 in the housing 12 in the pre-delivery or storage state, moving the guard member 32 from the extended position to the retracted position, e.g., by pressing the distal end of the guard member 32 against the patient's skin at the injection site, may result in the insertion end 28 of the delivery member 16 being inserted into the patient's skin.

The guard mechanism may further include a guard biasing member 35 and a guard extension 37. The guard extension 37 may be positioned proximal to the guard member 32; and the guard biasing member 35 may be positioned proximal to the guard extension 37. The guard extension 37 may have a hollow and generally cylindrical or tubular shape centered about the longitudinal axis A. Furthermore, the guard extension 37 may be moveable in a linear direction along the longitudinal axis A relative to the housing 12. In the present embodiment, the guard extension 37 is a separate structure from the guard member 32. However, in alternative embodiments, the guard extension 37 and the guard member 32 may be integrally formed in one piece to define a single, monolithic structure. In such alternative embodiments, the proximal end of the guard member 32 may correspond to the guard extension 37.

The guard biasing member 35 may be positioned between and in contact with the guard extension 37 and a releaser member 52. The guard biasing member 35 may be configured to bias or urge the guard extension 37 in the distal direction and bias or urge the releaser member 52 in the proximal direction. The guard biasing member 35 may initially be in an energized (e.g., compressed) state such that it exerts a biasing force on the guard extension 37 and a biasing force on the releaser member 52 in the pre-delivery state. In some embodiments, a distal end of the guard extension 37 is initially in contact with a proximal end of the guard member 32, as seen in FIG. 2. As a consequence, the guard extension 37 transfers a biasing force of the guard biasing member 35 to the guard member 32, such that the guard biasing member 35 biases or urges the guard member 32 toward the extended position. A user may overcome the biasing force by pressing the guard member 32 against the injection site. In doing so, the guard member 32 and the guard extension 37 move jointly in the proximal direction until, for example, the guard member 32 reaches the retracted position. When the injection is complete and the drug delivery device 10 is lifted off of the injection site, the guard biasing member 35 may push the guard extension 37 so that the guard extension 37 and the guard member 32 move jointly in the distal direction. This motion returns the guard member 32 to the extended position, which has the effect of covering the insertion end 28 of the deliver member 16. In some embodiments, the guard biasing member 35 may include a compression spring (e.g., a helical compression spring). Furthermore, in embodiments where the plunger biasing member 50 also includes a compression spring, the guard biasing member 35 may disposed around and/or have a larger diameter than the plunger biasing member 50.

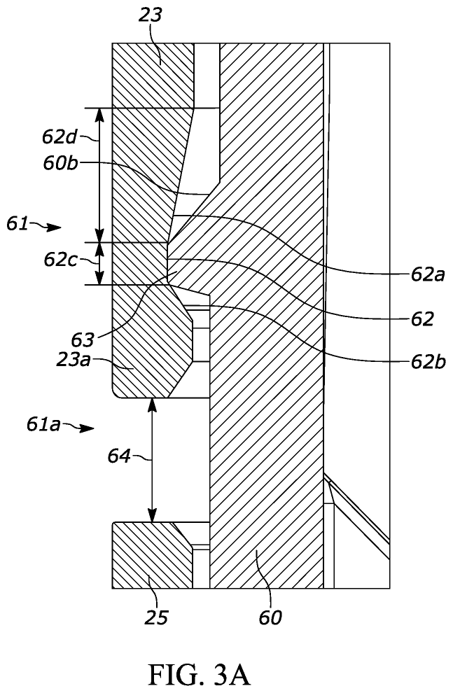

After drug delivery is complete and the guard member 32 has been re-deployed to the extended position, it may be desirable to lock the guard member 32 in the extended position to prevent subsequent user contact with the insertion end 28 of the delivery member 16 and/or to prevent re-use of the drug delivery device 10. Pursuant to these ends, some embodiments of the drug delivery device 10 may include a lock ring 40 configured to selectively rotate, depending on the axial position of the guard member 32, in order to lock the guard member 32 in the extended position once the guard member 32 has moved from the retracted position to the extended position. In the present embodiment, the lock ring 40 is centered and rotates about the longitudinal axis A. As illustrated in FIG. 3, a proximal end of the lock ring 40 may be in contact with the container holder 33 and the distal end of the lock ring 40 may be disposed at least partially within the guard member 32. The lock ring biasing member 51 may be positioned in the axial direction between a distally facing surface of the lock ring 40 and a proximally facing surface of the guard member 32. The lock ring biasing member 51 may initially be in a compressed or energized state such that it biases the lock ring 40 and the guard member 32 away from each other. As such, the lock ring biasing member 51 may exert a biasing force urging the guard member 32 toward the extended position, as well as exert a biasing force urging the proximal end of the lock ring 40 against the container holder 33. In some embodiments, the lock ring biasing member 51 may include a compression spring (e.g., a helical compression spring). In some embodiments, rotation of the lock ring 40 may be achieved by a camming arrangement between the lock ring 40 and the container holder 33.

The drug delivery device 10 may further include a drive mechanism 30 disposed partially or entirely within the housing 12. Generally, the drive mechanism 30 may be configured to store energy and, upon or in response to activation of the drive mechanism 30 by the user, release or output that energy to drive the plunger 26 to expel the drug 22 from the drug storage container 20 through the delivery member 16 into the patient. In the present embodiment, the drive mechanism 30 is configured to store mechanical potential energy; however, alternative embodiments of the drive mechanism 30 may be configured differently, for example, with the drive mechanism 30 storing electrical or chemical potential energy. Generally, upon activation of the drive mechanism 30, the drive mechanism 30 may convert the potential energy into kinetic energy for moving the plunger 26.

In the present embodiment, the drive mechanism 30 includes the plunger biasing member 50, a plunger biasing member seat 38, the releaser member 52, and a plunger guide 60. The plunger biasing member 50 may include a compression spring (e.g., a helical compression spring) which is initially retained in an energized state. In the energized state, the plunger biasing member 50 may be compressed such that its axial length is shorter than it would be in a natural or de-energized state. When released, the plunger biasing member 50 may try to expand to its natural axial length, and as a consequence, exert a biasing force pushing the plunger 26 in the distal direction.

The plunger biasing member 50 may be disposed at least partially within the plunger 26, and may have a distal end abutting against a proximally facing inner surface of the plunger 26 and/or may be fixedly attached to an inner surface of the plunger 26. So that the plunger biasing member 50 may be received within the plunger 26, an outer diameter or other dimension of the plunger biasing member 50 may be equal to or less than an inner diameter of the a ring 45 and/or equal to or less than an inner diameter of the hollow rod 46. In some embodiments, the distal end of the plunger biasing member 50 may abut against a proximally facing inner surface of the base 47 of the plunger 26. Furthermore, a proximal end of the plunger biasing member 50 may abut against a distally facing surface of the plunger biasing member seat 38. The plunger biasing member seat 38 may be fixedly attached to the tubular housing 25 such that the plunger biasing member seat 38 provides a stationary surface for the plunger biasing member 50 to push off of. So configured, the plunger biasing member 50, when released from the energized state, may expand in length with distal end of the plunger biasing member 50 moving in the distal direction away from the stationary proximal end of the plunger biasing member 50. This motion may push the plunger 26 is the distal direction, which, in turn, may push the stopper 24 in the distal direction to expel the drug 22 from the drug storage container 20 into the delivery member 16 and thereafter into the patient.

The releaser member 52 may have a hollow and generally cylindrical or tubular shape, and may be centered about the longitudinal axis A. As illustrated in FIG. 2, the releaser member 52 may be positioned in the radial direction between the distal end of the plunger guide 60 and a proximal end of the guard extension 37. Furthermore, the releaser member 52 may be arranged radially inwardly of the guard biasing member 35. Generally, the releaser member 52 is configured to operably couple the guard member 32 and the plunger 26 in an activation sequence and to generate an audible signal indicating the end of drug delivery. So configured, the releaser member 52 is exploited to perform two separate functions, and thus reduces the number of moving parts required by the drug delivery device 10.

The releaser member 52 may be configured to rotate relative to the housing 12 and/or translate linearly relative to the housing 12, depending on the stage of operation of the drug delivery device 10. Initial rotation of the releaser member 52 associated with activation may be powered by the plunger biasing member 50 and/or the guard biasing member 35; whereas later rotation of the releaser member 52 associated with generation of the end-of-dose signal may be powered solely by the guard biasing member 35. Any linear translation of the releaser member 52 without rotation may be powered solely by the guard biasing member 35. In some embodiments, the releaser member 52 may translate linearly only in the proximal direction; however, alternative embodiments may permit linear translation of the releaser member 52 in both the proximal and distal directions.

An ability of the releaser member 52 to rotate about the longitudinal axis A may be regulated by an interaction between an outer portion of an annular wall of the releaser member 52 and an inner portion of the guard extension 37. The guard extension 37 may be prevented from rotating about the longitudinal axis A as a consequence of its coupling to the housing 12. This has the effect of preventing rotation of the releaser member 52 about the longitudinal axis A when abutment structures (e.g., outwardly extending projections) included on the outer portion of the releaser member 52 engage cooperating abutment structures (e.g., inwardly extending projections) included on the inner portion of the guard extension 37. If the releaser member 52 is unable rotate, an outwardly extending projection of the plunger 26 received in a recess formed in the inner surface of the releaser member 52 is also unable to rotate. If this projection on the plunger 26 cannot rotate, then it cannot slide into a longitudinal opening in the plunger guide 60. If the projection cannot move in this manner, then plunger 26 also cannot move. If the plunger 26 cannot move, the plunger biasing member 50 cannot expand and de-energize. Thus, the releaser member 52 retains the plunger biasing member 50 in the energized state until the guard extension 37 moves to an axial position where the cooperating abutment structures on the outer portion of the releaser member 52 and the inner portion of the guard extension 37 disengage from each and thereby permit the releaser member 52 to rotate relative to the guard extension 37.

As discussed above, the removable cap 19 may have a storage position (FIGS. 1 and 3) where the removable cap 19 is coupled with the housing 12 and a removed position where the removable cap 19 is removed from and not coupled with the housing 12. As also discussed above, the device 10 may include a removable sterile barrier 21 that is removed from the delivery member 16 when the removable cap 19 is removed from the housing 12. The removable sterile barrier 21 may have a relatively snug or relatively high-friction fit with the drug storage container 20 to maintain the sterility of the delivery member 16 and/or to prevent air from entering the drug storage container 20. For example, in order to reduce the likelihood of contamination and/or occlusions or evaporated drug, it may be desirable to prevent or reduce the likelihood of air entering the drug storage container and/or the delivery member 16. Additionally or alternatively, it may be desirable to have a relatively snug or relatively high-friction fit between the sterile barrier 21 and the drug storage container 20 to prevent or reduce the likelihood of inadvertent needle sticks. For these or other reasons, it may also or alternatively be desirable to have a relatively snug or relatively high-friction fit between the removable cap 19 and the housing 12. The sterile barrier 21 and the removable cap 19 may also be coupled with their respective components (e.g., drug storage container 20 and housing 12) via other suitable features, such as coupling tab/slot connections, breakable connections such as perforated seals, threaded connections, or other features that achieve relatively secure but removable connections between respective components.

As a result of these coupling forces, features, and/or other factors, some device users may experience difficulty or discomfort removing the removable cap 19. As an example, some device users may have difficulty removing the cap 19 via axial forces alone (along longitudinal axis A). In other words, some device users may have difficulty in pulling the cap 19 off of/away from the housing 12. The cap 19 shown in Figs. FIGS. 1-3 includes a plurality of ribs 19d to help the user grip the surface of the removable cap 19 when removing the same.

The device 10 shown in FIGS. 1-3 also includes camming features to translate rotational motion into axial motion such that, upon rotational movement of the removable cap 19, the removable cap 19 is urged away from the housing 12, thereby facilitating and/or easing removal of the cap 19. For example, the housing 12 includes a housing camming feature 12a and a cap camming feature 19c. As a more specific example, to remove the removable cap 19 from the housing 12 via axial force/movement only (e.g., “straight-pull force”), a user may be required to exert 45 Newtons or less; approximately 40 to 45 Newtons; approximately 35 to 40 Newtons; approximately 30 to 35 Newtons; approximately 25 to 30 Newtons; approximately 20 to 25 Newtons; approximately 15 to 20 Newtons; approximately 10 to 15 Newtons; approximately 5 to 10 Newtons; or less than approximately 5 Newtons. In the device 10 shown in FIGS. 1-3, removing the removable cap 19 requires approximately 10 to 15 Newtons of straight-pull force.

The cap camming feature 19c shown in FIGS. 1-3 defines a wave shape, such as an arc-shaped surface. As a more specific example, the removable cap 19 shown in the figures includes a generally cylindrical body portion 19d and an end wall 19e that is generally perpendicular to the body portion 19d at the distal end of the cap 19. The body portion 19d defines a generally annular leading rim 19f at the proximal end of the cap 19. The leading rim 19f defines the wave shaped cap camming feature 19c. As an even more specific example, the leading rim 19f shown in the figures defines two wave shaped camming surfaces 19c and two relatively flat surfaces 19c′ that extend between wave shaped camming surfaces 19c. In other words, the two wave shaped camming surfaces 19c and the two relatively flat surfaces 19c′ cooperate to define the leading rim 19f. Alternatively, the leading rim 19f may define a continuous wave shape such as a continuous sinusoidal wave or another continuous wave shape. For the purposes of this application, the term “continuous” should be interpreted to mean that the wave shape continues around the entire perimeter of the leading edge rather than alternating wave shaped and flat surfaces

The housing camming feature 12a shown in FIGS. 1-2 defines a wave shape, such as an arc-shaped protrusion extending away from the outer surface 25 of the housing 12. As a more specific example, the housing camming feature 12a is a protrusion having a shape that is not unlike a “smile” or a “crescent moon” shape. As an even more specific example, the housing 12 shown in the figures defines two wave shaped camming features 12a.

When the removable cap 19 is in the storage position 19a shown in FIGS. 1-2, the cap camming features 19c engage or abut the housing camming features 12a. Additionally, the respective camming features 12a, 19c shown in the figures have matching or mirrored shapes such that the respective surfaces 12a, 19c slide smoothly/easily across each other. For example, when the removable cap 19 is rotated (either clockwise or counterclockwise) with respect to the housing 12, the housing camming features 12a, 19c rotate with respect to each other and urge the removable cap 19 away from the housing 12 along axis A. In other words, the camming features 12a, 19c translate rotational motion into axial motion to remove or assist with removal of the cap 19. In some embodiments, even a relatively small rotation may facilitate and/or ease removal of the cap 19.

Having described the general configuration of the drug delivery device 10, a general method of using the drug delivery device 10 to perform an injection will now be described. As a preliminary step, the user may remove the drug delivery device 10 from any secondary packaging, such as a plastic bag and/or cardboard box. Also, as a preliminary step, the user may prepare the injection site, e.g., by rubbing the patient's skin with an alcohol wipe. Next, the user may pull and detach the removable cap 19 from the housing 12, as described below in more detail. As a result of this motion, the gripper 13 may pull and detach the removable sterile barrier 21 from the drug storage container 20. This may uncover the insertion end 28 of the delivery member 16. Nevertheless, the insertion end 28 of the delivery member 16 will remain surrounded by the guard member 32 at this stage because the guard member 32 is arranged in the extended position. Next, the user may position the drug delivery device 10 over the injection site and then push the distal end of the guard member 32 against the injection site. The force applied by the user will overcome the biasing force of the guard biasing member 35 and the biasing force of the lock ring biasing member 51, thereby causing the guard member 32 to retract into the opening 14 moving from the extended position to the retracted position in the proximal direction. The delivery member 16 remains stationary relative to the housing 12 during the retracting movement of the guard member 32.

Movement of the guard member 32 from the extended position to the retracted position may cause several actions to occur. Because the delivery member 16 remains stationary relative to the housing 12 during retraction of the guard member 32, the insertion end 28 of the delivery member 16 is caused to extend through an opening in the distal end of the guard member 32, thereby piercing the patient's skin at the injection site and penetrating into the patient's subcutaneous tissue. In addition, retraction of the guard member 32 may also activate the drive mechanism 30 to expel the drug 22 from the drug storage container 20.

When the guard member 32 moves from the extended position to the retracted position, the guard member 32 may push the guard extension 37 in the proximal direction. During proximal movement of the guard extension 37, the above-mentioned cooperating abutment structures on the outer portion of the releaser member 52 and the inner portion of the guard extension 37 may slide past one another until they are no longer in contact with one another. When that occurs, the releaser member 52 may be free to rotate about the longitudinal axis A. Rotation of the releaser member 52 at the present stage is caused by the plunger biasing member 50 expanding and pushing a distally facing camming surface included in on the plunger 26 to slide along a proximally facing camming surface on the plunger guide 60. The resulting camming action causes the plunger 26 to rotate, which, in turn, may cause the releaser member 52 to jointly rotate.

Joint rotation of the releaser member 52 and the plunger 26 may continue until the distally facing camming surface included in on the plunger 26 reaches the end of the proximally facing camming surface on the plunger guide 60 and moves into a longitudinal slot formed in the plunger guide 60. The longitudinal slot does not inhibit linear movement of the plunger 26. As consequence, the plunger 26 is driven by the expanding plunger biasing member 50 to translate linearly in the distal direction. As a consequence, the plunger 26 comes into contact with the stopper 24 (if it is not already in contact with the stopper 24) and thereafter pushes the stopper 24 in the distal direction to expel the drug 22 from the drug storage container 20 through the delivery member 16 and out of the insertion end 28 into the patient's tissue. Drug delivery may carry on until the stopper 24 reaches the end-of-dose position. Here, the stopper 24 may abut against a proximally facing portion of the interior surface 15 of the wall of the drug storage container 20. As a result, the plunger 26 ceases moving in the distal direction.

After delivery is complete, the user may then lift the drug delivery deice 10 off of the injection site. With nothing to resist it, the guard biasing member 35 may push the guard member 32 from the retracted position to the extended position to cover the insertion end 28 of the delivery member 16. In some embodiments, this movement of the guard member 32 may cause the lock ring 40 to rotate to a position where it prevents subsequent retraction of the guard member 32.

These and other aspects of an exemplary drug delivery device are discussed in more detail in U.S. patent application Ser. No. 17/036,690, filed Sep. 29, 2020, U.S. patent application Ser. No. 17/035,851, filed Sep. 29, 2020, U.S. patent application No. 17/035,927, filed Sep. 29, 2020, U.S. patent application Ser. No. 17/036,129, filed Sep. 29, 2020, U.S. patent application Ser. No. 17/036,217, filed Sep. 29, 2020, and U.S. Provisional Patent Application entitled “DRUG DELIVERY DEVICE” filed by Applicant of the present application on the same day as the present application, the entire contents of each of which are incorporated by reference.

As described above, it may be advantageous to incorporate one or more shock absorbing features into the rear cap 23. In the event that the drug delivery device 10 is accidentally dropped from a height such that the rear cap contacts the ground with substantial velocity or the removable cap otherwise strikes or is struck with an external object with substantial velocity, the rear cap may experience substantial impulse force(s). Without shock absorbing features, such impulse forces may be transferred to other components within the device 10. Such force(s) have the potential to trigger the activation of automated or semi-automated features included in the drug delivery device 10 and/or cause damage to the drug delivery device 10. As an example, dropping the drug delivery device 10 with the longitudinal axis A parallel or substantially parallel to the direction of gravity and with the rear cap 23 facing generally downwards may, due to the deceleration associated with the drug delivery device 10 striking the ground, cause the releaser member 52 to move proximally (towards the rear cap 23, in the upward direction in FIG. 2) and/or cause the guard member 32 to retract into the housing. Either or both of these exemplary movements may potentially trigger the drive mechanism 30, thereby causing an unintended and/or premature injection. Additionally or alternatively, the deceleration may cause the lock ring 40 to rotate or otherwise move to a position where it prevents subsequent retraction of the guard member 32. This, in turn, may prematurely lockout of the guard member 32, thereby preventing a user from using the drug delivery device 10 to perform an injection.

As a more specific example of potentially undesirable consequences of dropping the drug delivery device 10, if the device 10 is dropped with the rear cap 23 facing downwards, most or all of the components of the device 10 are traveling and accelerating at roughly the same rate, but when the rear cap 23 impacts the ground or other surface then the housing 12 will decelerate before or at a faster than other internal components, such as the releaser member 52 and/or the guard member 32. In other words, upon impact, the housing will abruptly stop falling and have a relatively large deceleration while some of the other internal components are still traveling and/or accelerating towards the ground. As a result of the relatively large difference between the deceleration of the housing and the acceleration of other internal components (“acceleration delta”), upon impact or momentarily thereafter, the releaser member 52 and/or the guard member 32 may move proximally (towards the rear cap 23, in the upward direction in FIG. 2) within the housing 12, thereby potentially triggering the injection sequence. However, the shock absorbing features described herein may reduce the rate at which the housing decelerates upon impact, thereby reducing the acceleration delta between respective components such as the housing 12 on one hand and the releaser member 52 and/or the guard member 32 on the other hand and reducing the likelihood of a premature or unintended activation.

As another example of potentially undesirable consequences of dropping the drug delivery device 10, if the device 10 was recently removed from cold storage (e.g., a temperature at 10° C. or lower, at 5° C. or lower, or at 0° C. or lower) prior to being dropped, there may be a risk, due to, e.g., the reduced elasticity of certain materials at low temperatures, for component of the drug delivery device 10 to fracture or crack. Such fractures or cracks may compromise proper operation of the drug delivery device 10, and, even if they do not, if they are visible to the user, they may cause the user to assume that the drug delivery device 10 is defective and consequently discard the drug delivery device 10, which may or may not be necessary.

The reaction forces described above, if applied to the shock absorber, may cause conversion of kinetic energy into another form of energy such as thermal energy (e.g., heat) and/or spread out the time of an impulse. This, in turn, may reduce the likelihood of the impact event causing activation of automated or semi-automated features included in the drug delivery device including, for example, a drive mechanism for expelling a drug and/or a guard locking mechanism and/or reduce the likelihood of structural damage to components of the drug delivery device, including, for example, rear cap. In at least some scenarios, the rear cap during an impact event may function as a spring-and-damper system and/or a shock absorber.

Turning to FIG. 3, an exemplary embodiment of the above-mentioned shock absorber will now be described. The device 10 includes a shock absorber 61 configured to absorb an impact force and prevent unintended movement of the releaser member 52. In the device shown in FIG. 3, the shock absorber includes the rear cap 23 and the tubular housing 25 which are operably coupled with each other such as to permit relative movement therebetween and absorb impact force. As a more specific example, the shock absorber 61 includes a snap ring 62 configured to permit relative movement between the rear cap 23 and the tubular housing 25. In the device shown in FIG. 3, the snap ring 62 is an annular ring defined by an inner wall portion of the rear cap 23. Although the snap ring may be concave, convex, or another suitable shape, the snap ring 62 shown in FIG. 3 is generally concave such as to operably couple with an annular ridge 63. The annular ridge 63 shown in FIG. 3 is defined by an outer wall portion of the plunger guide 60, but the annular ridge 63 may be defined by other components such as the tubular housing 25, another component of the housing 12, or another suitable component. The snap ring 62 and the annular ridge 63 are configured to have a first position 61a (shown in FIG. 3A) when no external impact forces are being applied or after-effects thereof are being felt, such as when the device 10 is in its storage state, pre-delivery state, delivery or dosing state, or post-delivery state. In this first position, the rear cap 23 is spaced apart from the tubular housing 25, along the longitudinal axis A, by a buffer gap 64 that permits the rear cap 23 to move in the distal direction (downward in FIG. 3A).

The rear cap 23 is defined by a generally cylindrical side wall 23a and a generally convex top wall 23b. The side wall 23a shown in FIGS. 1-3 is flexible enough to allow the sidewall to flex radially outwardly during application of the shock absorber. The side wall 23a of the snap ring 62 is further defined by first and second ramped surfaces 62a and 62b that interact with the annular ridge 63 to maintain the rear cap 23 in the first position 61a unless external forces are present. As a more specific example, the first ramped surface 62a is a distally-facing frustoconical shaped surface configured to permit the rear cap 23 to move in the distal direction (downward) upon application of an impact force and to then urge the rear cap 23 to move in a proximal direction (upward) after dissipation of the impact force. During this movement, the side wall 23a flexes radially outwardly like a spring and/or dampener. The shape and angle of the first ramped surface 62a is configured to allow the rear cap 23 to permit movement in the distal direction (downward in FIG. 3A) during an impact event and then to urge the rear cap 23 in the proximal direction (upward in FIG. 3A) after the impact and after-effects of the same. As a more specific example, if the device is dropped or otherwise impacted, then the snap ring 62 may be urged into a second position 61b (shown in FIG. 3B), whereupon the first ramped surface 62a may urge the rear cap 23 back into the first position 61a when the impact forces and immediate effects thereof have dissipated or ended. As another more specific example, the rear cap 23 is configured to permit movement thereof without plastically deforming the rear cap 23 and/or causing the rear cap 23 to become stuck in the second position 61b. As an even more specific example, the rear cap 23 may be configured to prevent or reduce the likelihood of the releaser member 52 and/or the guard member 32 moving a distance during an impact sufficient to activate the device 10. The impact force may be similar or the same as a force caused by a drop from a height of 0.5 to 0.7 meters; by approximately 0.7 to 0.9 meters; by approximately 0.9 to 1.0 meters; by approximately 1.0 to 1.1 meters; by approximately 1.1 to 1.2 meters; by approximately 1.2 to 1.3 meters; by approximately 1.3 to 1.4 meters; by approximately 1.4 to 1.5 meters; by approximately 1.5 to 1.7 meters; by approximately 1.7 to 2.0 meters; or another suitable height. As another exemplary specification, the device may be activated upon a longitudinal movement by the releaser member 52 of approximately 7 to 8 mm; of approximately 6 to 9 mm; of approximately 5 to 10 mm; of approximately 4 to 11 mm; of approximately 3 to 12 mm; of approximately 2 to 15 mm; or another suitable distance. As another exemplary specification, the device may be activated upon a longitudinal movement by the guard member 32 of approximately 10 to 11 mm; of approximately 9 to 12 mm; of approximately 8 to 13 mm; of approximately 7 to 14 mm; approximately 6 to 15 mm; of approximately 5 to 16 mm; approximately 3 to 18 mm; or another suitable distance.

The rear cap 23 may be configured to have a side wall 23a flexible enough to move upon impact but stiff enough to spring back into the first position after impact and the after-effects thereof. Also, the second ramped surface 62b is a proximally-facing frustoconical shaped surface configured to allow the rear cap 23 to slide over the annular ridge 63 during assembly and then resist or prevent removal of the rear cap 23 after assembly.

The snap ring 62 may have a longitudinal height 62c generally equal to the longitudinal height of the annular ridge 63 to hold the components in the first position during normal use. The first ramped surface 62a may have a longitudinal height 62d generally equal to the longitudinal height of the buffer gap 64.

The side wall of the rear cap may be a continuous cylinder shape or it may have one or more interrupted portions that permit or facilitate elastic deformation/flexure during impact. For example, the side wall may include one or more slits formed therein adjacent to the distal end thereof. The slit(s) may extend partially or completely through the side wall and may extend a portion or the full length of the wall. The slit(s) may extend generally parallel with the longitudinal axis or along another direction/ orientation.

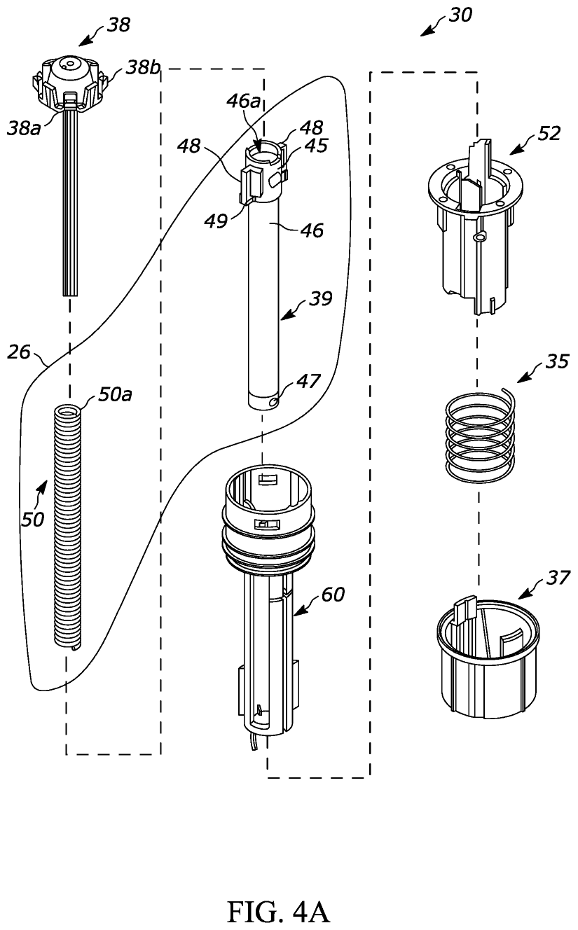

Turning to FIG. 5, the rear cap 23 is shown in full and in isolation (rather than installed in the device). The rear cap 23 includes the side wall 23a, the convex top wall 23b, and the snap ring 62 discussed above. The rear cap 23 also includes a plurality of longitudinal ribs 23c that provide a hard stop for the annular ridge 63 shown in the prior figures. For example, the longitudinal ribs resist or prevent the rear cap 23 from traveling distally past the point where the annular ridge abuts the longitudinal ribs 23c. The longitudinal ribs may also provide strength or stiffness for the rear cap 23. The first ramped surface 62a forms an angle 23d with respect to the longitudinal axis A and the angle 23d may be approximately 20 degrees; approximately 18 to 22 degrees; approximately 16 to 24 degrees; approximately 14 to 26 degrees; approximately 12 to 28 degrees; approximately 10 to 30 degrees; approximately 5 to 35 degrees; approximately 5 to 40 degrees; approximately 5 to 45 degrees; or another suitable angle.

As an alternative or additional form of impact absorption, the rear cap 23 may be able to deform radially due to interaction between the and the plunger guide 60. As an example, the rear cap 23 shown in FIG. 5 includes three ribs 23c so the rear cap 23 may be deformed radially outwardly in the areas near the respective ribs 23c. As a result, the areas between the ribs 23c may be deformed radially inwardly. In other words, the rear cap 23 shown in FIG. 5 may be deformed to become more of a rounded-triangular shaped, with the three “points” of the rounded-triangle being aligned with the ribs 23c. If the rear cap 23 has a different number of ribs, such as two, four, five, six, or any other suitable number, then the deformed shape may correspond accordingly.

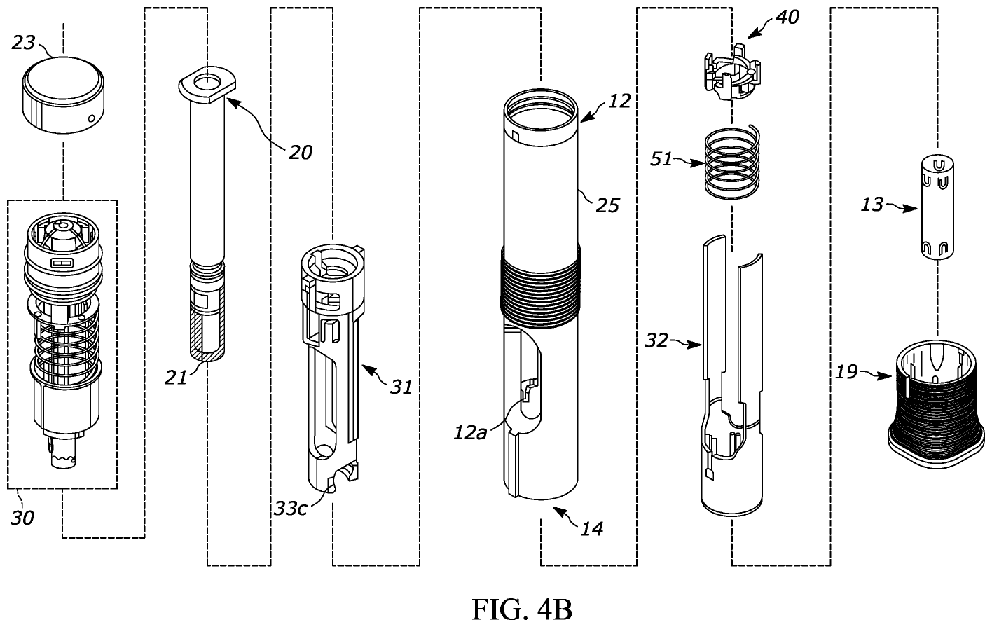

Turning to FIG. 6, another embodiment of a rear cap 123 will now be described. Various elements of the rear cap 123 illustrated in FIG. 6 may be similar or identical in structure, configuration, and/or function to elements of the rear cap 123 described above in conjunction with FIGS. 1-5. Such elements are assigned with the same reference numeral as used in FIGS. 1-5, except incremented by 100 or a multiple thereof. A description of some of these elements is abbreviated or eliminated in the interest of conciseness. The rear cap 123 includes the side wall 123a, the convex top wall 123b, and the snap ring 162 discussed above. The rear cap 123 also includes a plurality of longitudinal ribs 123c that provide a hard stop for an annular ridge similar to that shown in the prior figures. For example, the longitudinal ribs resist or prevent the rear cap 123 from traveling distally past the point where the annular ridge abuts the longitudinal ribs 123c. The longitudinal ribs may also provide strength or stiffness for the rear cap 123. The first ramped surface 162a forms an angle 123d with respect to the longitudinal axis A and the angle 123d may be approximately 10 degrees; approximately 8 to 12 degrees; approximately 6 to 14 degrees; approximately 4 to 16 degrees; approximately 3 to 18 degrees; approximately 3 to 20 degrees; approximately 3 to 25 degrees; approximately 3 to 30 degrees; approximately 3 to 35 degrees; or another suitable angle. The rear cap 123 also includes at least one hard stop such as a plurality of hard stops 123e that define a maximum distance the rear cap 123 can travel with respect to one or more components of the device, such as a plunger guide similar to that shown in the prior figures.

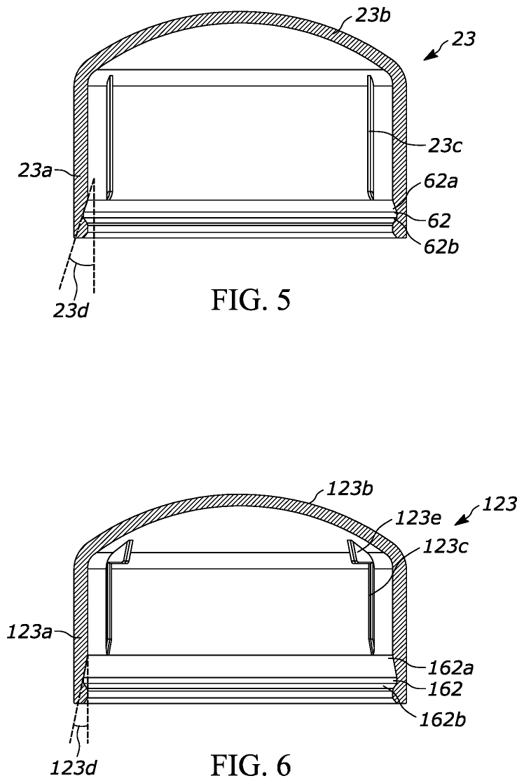

Turning to FIG. 7, another embodiment of a rear cap 223 will now be described. Various elements of the rear cap 223 illustrated in FIG. 7 may be similar or identical in structure, configuration, and/or function to elements of the rear caps described above. Such elements are assigned with the same reference numeral as used in FIGS. 1-6, except incremented by 200 or a multiple thereof. A description of some of these elements is abbreviated or eliminated in the interest of conciseness. The rear cap 223 includes the side wall 223a, the convex top wall 223b, and the snap ring 262 discussed above. The rear cap 223 also includes a plurality of longitudinal ribs 223c that provide a hard stop for an annular ridge similar to that shown in the prior figures. For example, the longitudinal ribs resist or prevent the rear cap 223 from traveling distally past the point where the annular ridge abuts the longitudinal ribs 223c. The longitudinal ribs may also provide strength or stiffness for the rear cap 223. The first ramped surface 262a forms an angle 223d with respect to the longitudinal axis A and the angle 223d may be approximately 20 degrees; approximately 18 to 22 degrees; approximately 16 to 24 degrees; approximately 14 to 26 degrees; approximately 12 to 28 degrees; approximately 10 to 30 degrees; approximately 5 to 35 degrees; approximately 5 to 40 degrees; approximately 5 to 45 degrees; or another suitable angle. The rear cap 223 also includes at least one hard stop such as a plurality of hard stops 223e that define a maximum distance the rear cap 223 can travel with respect to one or more components of the device, such as a plunger guide similar to that shown in the prior figures. The rear cap convex top wall 223b defines a thinner wall than the corresponding top wall in FIGS. 5 and 6. As a more specific example, the convex top wall 223b is approximately 0.5 mm thick, whereas the top wall shown in FIGS. 5 and 6 is approximately 0.7 mm thick. The rear cap 223 also includes a flow leader 223f to improve the injection molding process.

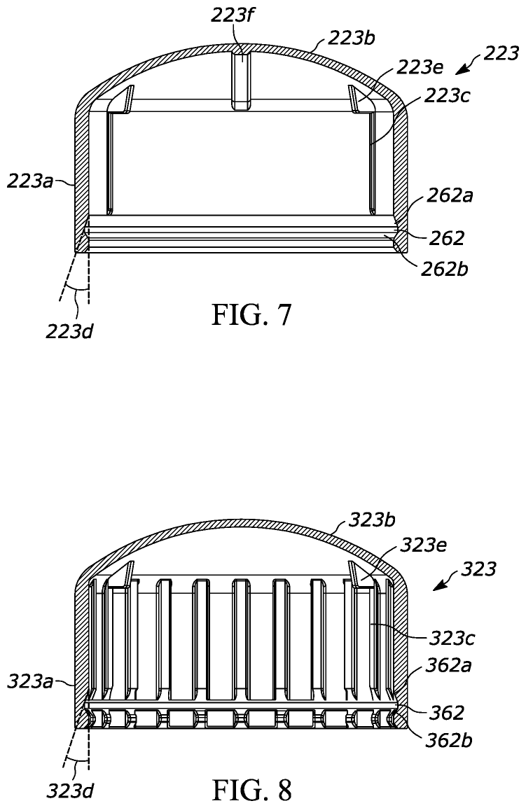

Turning to FIG. 8, another embodiment of a rear cap 323 will now be described. Various elements of the rear cap 323 illustrated in FIG. 8 may be similar or identical in structure, configuration, and/or function to elements of the rear caps described above. Such elements are assigned with the same reference numeral as used in FIGS. 1-7, except incremented by 300 or a multiple thereof. A description of some of these elements is abbreviated or eliminated in the interest of conciseness. The rear cap 323 includes the side wall 323a, the convex top wall 323b, and the snap ring 362 discussed above. The rear cap 323 also includes a plurality of longitudinal ribs 323c that cooperate to define the first ramped surface 362a. As a more specific example, the plurality of longitudinal ribs 323c are radially spaced from each other around the inner surface of the side wall 323a and each or many of the ribs 323c has a similar or same angle 323d with respect to the longitudinal axis A such that the plurality of ribs define a path for receiving an annular ridge similar to that shown in FIGS. 1-5. The angle 323d may be approximately 20 degrees; approximately 18 to 22 degrees; approximately 16 to 24 degrees; approximately 14 to 26 degrees; approximately 12 to 28 degrees; approximately 10 to 30 degrees; approximately 5 to 35 degrees; approximately 5 to 40 degrees; approximately 5 to 45 degrees; or another suitable angle. The rear cap 323 also includes at least one hard stop such as a plurality of hard stops 323e that define a maximum distance the rear cap 323 can travel with respect to one or more components of the device, such as a plunger guide similar to that shown in the prior figures. The rear cap convex top wall 323b defines a thinner wall than the corresponding top wall in FIGS. 5 and 6. As a more specific example, the convex top wall 323b is approximately 0.5 mm thick, whereas the top wall shown in FIGS. 5 and 6 is approximately 0.7 mm thick.

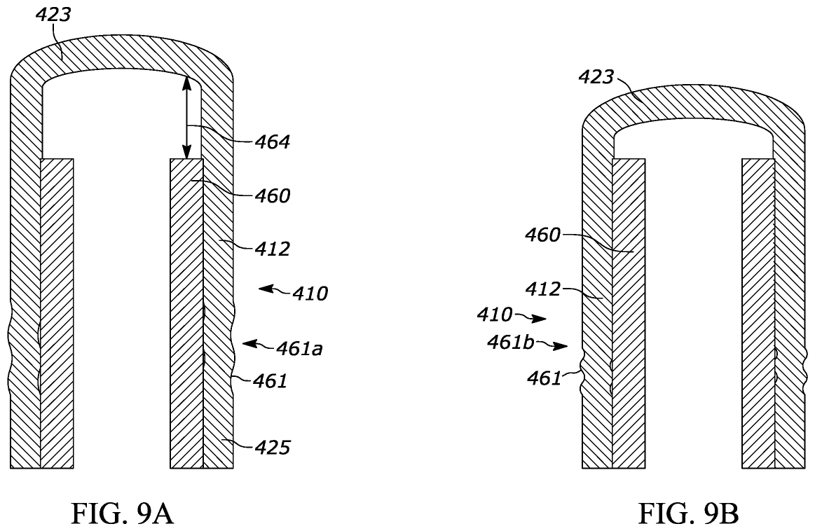

Turning to FIGS. 9A and 9B, another embodiment of shock absorber 461 for a device 410 will now be described. Various elements of the device 410 illustrated in FIGS. 9A and 9B may be similar or identical in structure, configuration, and/or function to elements of the rear caps described above. Such elements are assigned with the same reference numeral as used in FIGS. 1-8, except incremented by 400 or a multiple thereof. A description of some of these elements is abbreviated or eliminated in the interest of conciseness. The device 410 includes a housing 412 with a proximal portion (including at least a rear cap 423 and a tubular housing section 425) defining a single, monolithic structure and the shock absorber 461 includes a flexible or compressible portion coupling the tubular housing 425 and the rear cap 423. As a more specific example, FIG. 9A shows cross-sectional views of portions of a housing and plunger guide that may be utilized as part of a shock absorber in accordance with various aspects of a drug delivery device. FIG. 9A shows the shock absorber 461 having a first position 461a (shown in FIG. 9A) when no external impact forces are being applied or after-effects thereof are being felt, such as when the device 410 is in its storage state, pre-delivery state, delivery or dosing state, or post-delivery state. In this first position, the rear cap 423 is spaced apart from a plunger holder 460, along the longitudinal axis A, by a buffer gap 464 that permits the rear cap 23 to move in the distal direction (downward in FIG. 9A)., where the housing is in a first position where the device is not experiencing an impact event or the after-effects thereof and a second position 461b during an impact event or the after-effects thereof. The shock absorber 461 may act similar to a spring, permitting the housing 412 to axially compress during an impact and returning to a relaxed state thereafter (e.g., in the second position 461b). The flexible or compressible portion in the shock absorber 461 may be made of a thermoplastic material, an elastomeric material, a coil-spring covered in another material, or any other suitable configuration.

Any or all of the above-described shock absorbers may be utilized in a device having an outer label such as a plastic film containing information or labeling regarding the drug product and/or the drug delivery device. The label may be positioned over the shock absorber so that a patient or end user does not readily see the shock absorber. In such a case, the label may become temporarily or permanently wrinkled upon impact event(s).

All features disclosed herein with respect to any of the removable cap embodiments may be combined in any combination, except combinations where at least some of such features are mutually exclusive.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, VRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-a4integrin mAb); Valortim® (MDX-1303, anti-B. anthracis protective antigen mAb); ABthrax™, Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNFα monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1((R)-2-methyloxiran-2-yl)1-oxopentan-2-ylcarbamoyl)-2-phenylethyl)-2 -((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo- 1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1 x IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor vlll (EGFRvIII) BiTE® (bispecific T cell engager) molecule. In some embodiments, the drug delivery device may contain or be used with AMG 673 or another product containing a half-life extended (HLE) anti-CD33 x anti-CD3 BiTE® (bispecific T cell engager) construct. In some embodiments, the drug delivery device may contain or be used with AMG 701 or another product containing a half-life extended (HLE) anti-B-cell maturation antigen (BCMA) x anti-CD3 BiTE® (bispecific T cell engager) construct. In some embodiments, the drug delivery device may contain or be used with AMG 757 or another product containing a half-life extended (HLE) anti- delta-like ligand 3 (DLL3) x anti-CD3 BiTE® (bispecific T cell engager) construct. In some embodiments, the drug delivery device may contain or be used with AMG 910 or another product containing a half-life extended (HLE) epithelial cell tight junction protein claudin 18.2 x CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing defining a longitudinal axis and having an opening;
a drug storage container including a barrel, a stopper, and a delivery member, the stopper movably positioned within the barrel, the delivery member positioned at a distal end of the barrel and having an insertion end configured to extend at least partially through the opening during a delivery state;
a plunger moveable toward the distal end of the drug storage container to engage the stopper and expel a drug from the drug storage container through the delivery member;
a plunger biasing member coupled with the plunger and configured to urge the plunger toward the distal end of the drug storage container;
a releaser member having a first position wherein the releaser member prevents the plunger from moving into the delivery state and a second position wherein the releaser member does not prevent the plunger from moving into the delivery state; and
a shock absorber configured to absorb an impact force and prevent unintended movement of the releaser member.

2. The drug delivery device of claim 1, wherein the housing includes a tubular housing and a rear cap operably coupled with each other, and the shock absorber includes the rear cap.

3. The drug delivery device of claim 2, wherein the rear cap is movable with respect to the tubular housing.

4. The drug delivery device of claim 2, wherein the shock absorber includes a snap ring configured to permit relative movement between the rear cap and the tubular housing.

5. The drug delivery device of claim 4, wherein the shock absorber includes an annular ridge configured to be received by the snap ring.

6. The drug delivery device of claim 5, wherein the snap ring includes a ramped surface configured to permit the rear cap to move in a distal direction upon application of the impact force and to urge the rear cap to move in a proximal direction after dissipation of the impact force.

7. The drug delivery device of claim 6, wherein the ramped surface is defined by a plurality of longitudinal ribs.

8. The drug delivery device of claim 2, further comprising a buffer gap between the rear cap and the tubular housing.

9. The drug delivery device of claim 8, wherein the buffer gap defines a distance between the rear cap and the tubular housing.

10. The drug delivery device of claim 2, further comprising a plunger guide configured to operatively couple the tubular housing and the rear cap.

11. The drug delivery device of claim 10, wherein the plunger guide defines the annular ridge received by the snap ring of the rear cap and wherein the plunger guide further defines a second annular ridge configured to be received by a second annular ring.

12. The drug delivery device of claim 4, further comprising a plunger guide configured to operatively couple the tubular housing and the rear cap, wherein the plunger guide defines the annular ridge.

13. The drug delivery device of claim 2, wherein the drug delivery device is an autoinjector.

14. The drug delivery device of claim 2, wherein the tubular housing defines a generally cylindrical shape.

15. The drug delivery device of claim 2, wherein the tubular housing defines a non-cylindrical shape, such as a generally oblong or oval shape.

16. The drug delivery device of claim 2, wherein the tubular housing and the rear cap are defined by a single, monolithic structure and the shock absorber includes a flexible or compressible portion coupling the tubular housing and the rear cap.
